(19) United States
(12) Patent Application Publication
(10) Pub. No.: US 2002/0151467 A1
(43) Pub. Date: Oct. 17, 2002

(54) Methods and compositions for oral insulin delivery

(76) Inventor: Frank K. Leung, Washington, DC (US)

Correspondence Address: BARNES & THORNBURG, 2600 CHASE PLAZA, 10 LASALLE STREET, CHICAGO, IL, 60603

(21) Appl. No.: 09/746,855

(22) Filed: Dec. 21, 2000

(51) Int. Cl. A61K 38/28

(52) U.S. Cl. 514/4; 514/58

(57) ABSTRACT

Methods and compositions to deliver insulin to mammals in need thereof do not require injections but, because of special formulations, can be delivered orally or transdermally.

### BACKGROUND

Improved methods and compositions for delivery of insulin employ formulations suitable for oral delivery and routes that do not require injections.

Insulin is a large molecular weight protein required by diabetics to treat their disease. The molecule was discovered in 1922 by Banting and Best. Insulin was not successfully administered orally because it is destroyed by the digestive juices before it has a chance to work. Also, insulin is poorly absorbed through the mucous membrane of the gastrointestinal tract. As the present time, all insulin used in the treatment of diabetes is injected using a disposible syringe with an attached needle, a pen-cartridge device, or an infusion pump with a needle temporarily inserted into the skin. There are many forms of diabetes. Type 1 diabetics frequently require injections 4 or more times a day. Many Type 2 diabetics use insulin injections as well. New ways of delivering insulin are needed.

The incidence of diabetes is increasing globally. Indeed, the World Health Organisation predicts the number of diabetics will double by the year 2025 due to the increased number of elderly people who are at increased risk and to the rising incidence of obesity. According to the Pharmaceutical World Review 1999, the worldwide market for insulin was valued at $2.3 billion in 1998, showing 10% growth in local currency terms, over 1997. The North American market accounts for 39% of the total. In United States, there are at least 15 million known diabetics. 2 million require daily insulin injections, which is about 5% of the population. There are, additionally, several million undiagnosed diabetics. There are 150 million diabetics worldwide and this figure is expected to double to 300 million by the year 2025. Diabetes is the only major chronic disease with a significant increase in its prevalence over the years. In 1999, the market for diabetes care products was more than $6 billion USD. The insulin market now approaches $3 billion USD. Diabetes accounts for almost $100 billion in direct and indirect expenditures attributable to diabetes each year (AMA Diabetes Care, 1998)

Most of the Type 2 diabetics take oral agents which work on stimulating the pancreas to secrete insulin or enhance insulin action. The market would be greatly expanded if insulin could be given directly without the use of needles in Type 2 diabetics.

One of the reasons for poor diabetic control is the fact that patients are required to inject themselves every day. Not only does this reduce compliance, but it can also lead to complications. New delivery systems are needed, in particular because the incidence of diabetes is rising. It would be beneficial if diabetics were spared the inconvenience of injections.

Peripheral delivery of insulin by subcutaneous/intravenous/intramuscular/inhalation/injections is unphysiologic because it does not achieve the “first pass” effect of insulin in liver which is a critical step. The injections deliver insulin to muscles and fat before the hepatic “first-pass” can be achieved. In the natural physiology of a mammal, pancreatically-secreted insulin is delivered into the hepatic portal vein and is transported to liver, where significant glucose intake and insulin metabolism occur. The portal route of delivery was found to be superior to the peripheral mode of delivery in maintaining more appropriate insulin concentrations of hepatic glucose output (Canavan et al., 1997).

A clinically observed disadvantage of peripheral injections of insulin are hyperinsulinism which is known to be associated with hypertension, insulin resistance, cardiovascular diseases, acanthosis nigra, obesity, weight gain and peripheral edema.

The development of an oral-transmucosal or transdermal insulin would offer unmatched advantages over injections by:

1) Reducing the pain associated with injections.

2) Reducing the chance of skin infection and irritations.

3) Reducing the anxiety and trauma associated with injections.

4) Improve compliance.

5) Preventing the development of diabetes (treatment of low dose insulin orally in animals suggests this).

6) Improving the control of diabetes due to continuous absorptions and availability of insulin over many hours.

7) Avoiding the labile glucose responses comprising injections because oral or transdermal insulin may be more physiological.

Potential improved delivery systems include inhaled insulin, oral insulin, and transdermal insulin.

Each delivery system has its own difficulties in ensuring that an effective dose reaches the required tissues. However, inhaled insulin is likely to be the first used clinically (jointly developed by Pfizer and Hoechst Marion Roussel “HMR”). This formulation of insulin has been developed by Inhale Therapeutic Systems and is licensed to Pfizer. HMR is supplying the recombinant insulin.

Oral insulin, is currently in Phase II trials. Canada's Generex Biotechnology reportedly expects to launch its product during the year 2001. However, competition from transdermal insulin is likely to impact on inhaled and oral forms, because this is already in Phase I tests. Details on the development of alternative insulins can be found on IMS HEALTH's drug development database R&D focus. 10-year forecasts for insulin sales, taking into account future events such as new launches, are provided by IMS HEALTH's Pharmacast &Beyond therapy class forecasting system.

Oral Insulin

The development of an oral form of insulin is a challenge that has tested the scientific powers of the pharmaceutical industry for many years. A major problem is the fact that the digestive system metabolises insulin before it can reach the tissues. Despite this problem, many companies have decided to look for an oral form of insulin because this would help boost compliance and reduce complications. The need for daily injections is a major problem in diabetes. Companies that produce an easy-to-use alternative will be likely to benefit considerably, especially because the incidence of diabetes is rising each year.

One company, Canada's Generex Biotechnology, is conducting Phase II clinical trials with Oralin™. This is administered as a fine spray into the mouth using a hand-held aerosol. The insulin then passes into the bloodstream for distribution to the tissues. For patients this means an end to inconvenient injections and the avoidance of complications caused by multiple jabs. Reports of responses in the studies conducted so far that indicate this route of administration is effective and may provide a similar level of diabetic control as injected insulin. Generex has dosing trials in 60-80 diabetics, with a long-term safety and efficacy trial due to begin in up to 100 patients by October 1999. Oralin™ could be launched in 2001. Valentis (Nasdag:VLTS) announced on Aug. 17, 2000 the signing of a collaborative research agreement between Flemington Pharmaceutical (OTC Flem.ob) and Valentis' wholly-owned subsidiary, PolyMasc Pharmaceutical, to develop a formulation of PEGylated insulin spray.

The UK biotechnology company Cortecs is also developing an oral form of insulin called Macrulin™. This is in Phase II trials in Type II diabetics and has been reported to deliver biologically active insulin through intestinal absorption.

A US company Protein Delivery has just produced ‘proof of principle’ data for its oral insulin product, called M2. This is a modified form of insulin that is reported to effect glucose control.

Another oral form of insulin is being developed by the Irish drug delivery specialist Elan—this is in Phase I trials. Endorex also has a preclinical product available for licensing. Eli Lilly is conducting Phase II trials on the oral agent AI-401, developed by a US company AutoImmune. Details on the development of oral insulins can be found on IMS HEALTH's drug development database R&D focus.

Transdermal Insulins

One way of avoiding the problems of oral delivery of insulin is to put the hormone straight into the bloodstream. That is essentially what injections do. However, a viable alternative is to use transdermal technology, allowing the product to be absorbed directly into the capillaries of the skin. At first sight this is straightforward approach because many products are already delivered using transdermal patches. However, insulin is a large and complex molecule causing barriers to transmission transdermally. Helix BioPharma, from Canada, is reported to be developing a transdermal patch containing insulin. Using its proprietary BIPHASIX microencapsulation system, Helix has treated diabetic rats with transdermal insulin and claims to have significantly reduced blood glucose levels as a result.

In Germany, IDEA is conducting Phase I trials for a transdermal insulin that uses its Transfersomes drug delivery system. This is being tested at the WHO Diabetes Reference Centre in Dusseldorf.

Details on the development of transdermal insulins can be found on IMS HEALTH's drug development database R&Dfocus.

Inhaled Insulin

Inhaled forms of insulin could reach the marketplace by the end of 2001. Pfizer and Hoechst Marion Roussel have recently announced a co-operation to produce an inhaled insulin using technology from Inhale Therapeutic Systems that delivers a fine powder into the lungs. Phase III studies were started at more than 110 sites, for both Type I and Type II diabetes. Another inhaled insulin is being developed by Novo Nordisk and Aradigm. This is currently in Phase II trials and is expected to hit the market in 2003. It uses a different delivery method to the Inhale product and is breath-activated. Reports on studies to date are that when inhaled just before a meal, the Novo Nordisk system produces similar glucose control to an insulin injection.

A third inhaled version of insulin is in development with Eli Lilly and Dura Pharmaceuticals. This version uses Dura's Spiros, a battery-powered delivery system.

How to Compare Results of Different Research Data?

Percent reduction of initial glucose concentration is widely accepted as a measure of the pharmacodynamic effectiveness of insulin delivery (Dyschendra, 1998).

[image]

Factors Affecting Insulin Absorption Include:

1) Insulin structures:

a) Monomeric insulin is absorbed 3 times faster than natural human insulin.

b) The rate of disappearance is 2-3 times faster for dimeric and moneric insulin than regular human insulin.

2) Low bioavailabilty in perioral route insulin delivery:

a) Less than 0.5% of orally administered insulin is absorbed under the best conditions (Damge, 1997)

b) Proteolytic enzymes degrade most of the insulin.

c) Gastrointestinal transit time of insulin is highly variable.

d) Insulin is cleared by the “first-bypass” hepatic metabolism and clearance.

e) Insulin has a high molecular weight of about 6000 daltons.

Efforts to Improve Insulin Absorption Include:

1) Altering of insulin structures.

2) Using absorption promoters such as:

a) bile salts (glycocholate)

[glycocholic>deoxycholic>non-bile control>taurocholic>cholic acid]

b) sodium salicylate

c) azone—classical standard enhancer

d) soybean trypsin inhibitor

e) chitosan (polysaccharide)

3) Manipulating contact time:

a) viscosity control

b) bioadhesiveness control

4) Inhibiting enzyme degradation of insulin:

a) Proteolytic inhibitors

[Aprotinin >Bowman Bick inhibitor >Chromostatin and trypsin inhibitor]

b) Locked up enzymes.

5) Altering pH dependency:

a) insulin release depends on optimal pH

b) insulin absorption depends on optimal pH

6) Using nanospheres: insulin is loaded in poly(alkyl cyanoacrylate) nanospheres to protect from enzyme degradation, resulting in 50% drop in blood glucose

7) Manipulation of active and passive transport of insulin.

Despite a large amount of research and development in the field of insulin delivery to diabetics, no easy to use yet effective method is available.

### SUMMARY OF THE INVENTION

The present invention relates novel drug delivery methods and insulin compositions. In an embodiment, insulin is encapsulated by a polysaccharide (e.g. Cyclodextrin) forming a toroid drug complex containing the drug molecule inside. The complex prevents proteolytic degradation and aggregation of the drug. The complex dissociates rapidly after contact with water molecules and releases free drug to the diabetic patient. The cell membrane of the patent undergoes hydrotopic solubilization with exposed water binding sites on the cell membrane. Urea removes and moisturizes the membrane by exposing its water binding sites. The water molecules help to dissociate the drug complexes after a mucobioadhesive polymer such as carboxymethycellulose glues them to the cell surface. As result, there is a sudden increase in the concentration of free drug on the cell surface creating an enormous drug gradient which causes influx of the drug into the cells. Polymers also help to immobilize enzyme activity by locking them into some of the polymer covalent bonds. Carboxmethycellulose and some other polymers are also known to widen the cell junctions to allow high molecular weight molecules to go through the cells. Carboxymethycellulose is used as a lubricant for dry eyes. It attracts and retains moisture close to the cell membrane (Refresh Tears™ Allergan, Inc.). Other ingredients in the composition of the present invention include sodium lauryl sulfate, a surfactant, and hydroxypropyl methycellulose, a viscosity control polymer. Propylene glycol also moisturizes the cell membrane and acts as a solution stabilizer. Urea peroxide is a booster in the formulation because it dissociates itself into urea, water and oxygen molecules. Urea's peroxide further adds water molecules and exposes water binding sites at the same time. Its oxygen molecules also help some of the active transport of drugs through the ATP active transport system of the cell membrane.

The drug delivery applied to insulin delivery shows that the insulin formulations of the present invention are extremely effective. Insulin delivered orally achieves efficacy comparable to insulin delivered as injections, without the problems attending injections. The oral insulin concentration is 25 units/ml. Total oral insulin bioavailability is about 3-5% of that of insulin delivered by subcutaneous injection (at 5 min about 87%).

Insulin methods and compositions of the present invention have some clear-cut advantages over current modes of deliveries such as injection. Animal data disclosed herein indicated that Intesulin™ (oral insulin) delivered to the intestinal mucosa has a profound hypoglycemic effect and insulin response patterns which matched that in normal human physiology. These results predict a clear-cut improvement over the conventional injected or inhaled insulin delivery. (The animal insulin studies used Azone™ for standard comparison and control).

An experiment using oral and transdermal insulin delivery involved more than 258 animals (rats). Results showed that oral-transmucosal insulin absorption has peak absorption at 5 minutes compared to transdermal at 15-30 minutes. In controlled experiments, the subcutaneous injection had a peak insulin absorption at 60 minutes.

Formulations of the present invention have profound hypoglycemic effects of between 60 to 100% lasting for more than 5-8 hours depending on the dose and the site of delivery.

Preclinical data has shown that oral insulin of the present invention surpasses the injection reuslts:

1) Oral insulin has a peak insulin absorption at 5 minutes compareed to 60 minutes for subutaneous injection.

2) Insulin pharmacokinetics data showed that oral insulin is 20-30 times more sensitive in its hypoglyemic action than subcutaneous injections (as defined by AUC glucose divided by AUC insulin).

3) The oral insulin profile matches that in normal human physiology. Its peak absorption is 5 minutes and its insulin level returns to normal control level within 2 hours.

In summary, oral insulin provides a rapid onset and sustained hypoglycemic action without significant peripheral hyperinsulinemia. Preliminary data have showed that this method is more potent than subcutaneous insulin injection. Parenteral injection represents 100% bioavailability, but 99% of the injected insulin has gone to work in the wrong places like fat and muscles. This is why there are a lot of side-effects from peripheral hyperinsulinemia. “Bioavailability” should mean the efficacious bioavailability. Oral insulin of the present invention is delivered directly into the hepatic portal vein. If insulin sensitivity (or potency)=AUC glucose divided by AUC insulin absorbed, the oral insulin is many times more potent than subcutaneous injections.

Data on diabetic rats support that the methods and compositions of the present invention:

a) show excellent hypoglycemic potency in severe diabetic rats;

b) achieve comparable hypoglycemic potency to that achieved by subcutaneous injections;

c) can lower triglyceride blood levels;

d) severe diabetic animals have markedly increased absorption of Intesulin-B by several fold over non-diabetic animals. This suggests the uniqueness of the Intesulin formulation which improves absorption even in severe conditions of hypoglycemia and dehydration as occurred in diabetic rats. The blood insulin level is over 300% in diabetic rats compared to non-diabetic rats.

To the inventor's knowledge this is the first time such an interesting finding was ever reported.

This is expected to be an extremely attractive attribute in terms of drug delivery system—being capable of delivering drugs even in adverse conditions such as dehydration or in diabetes.

Definitions

Controls: receive no insulin.

C: means capsaicin is in the formation.

Injection Controls: receive insulin via injection.

NA: means a formulation without Azone, C or WG.

Trademark names of insulin compositions to be delivered to diabetics by methods disclosed herein, within the scope of the present invention, include:

1. Transdermal delivery: Dermulin™, Dermulin-C is Dermulin with Capsacin.

2. Oral-transmuocsal delivery: Musulin™

3. Intragastric delivery: Gastrisulin™; a gel capsule dissolves in the stomach and releases an insulin solution.

4. Intestinal delivery: Intesulin™; a gel capsule dissolves in the intestine and release an insulin solution.

All formations contain α-cyclodextrin except Intesulin-B with contains β-cyclodextrin.

All original formulations contain ∝-cyclodextrin at 0.1% concentration by weight. Alternatively, substitute, hydroxy β-cyclodextrin may be substituted at 0.5% concentration by weight ∝ is stronger, but is currently not FDA approved.

### BRIEF DESCRIPTION OF THE DRAWINGS

FIG. 1 shows in schematic form how the oral insulin compositions of the present invention works to deliver insulin to cells in need thereof; Stage 1 illustrates an insulin molecule complex with a carrier molecule; a cell membrane is shown with water molecules; Stage 2 shows a symbol (arrow) of a mucroadhesive polymer that helps attach the insulin complexes (2 arrows) to the cell surface membrane; Stage 3 illustrates that the insulin-carrier molecule dissociates, releasing insulin which concentrates on the cell surface (this concentration creates a sufficient gradient for the rapid transport of the insulin molecules across the cell membrane).

FIG. 2 is the % of blood glucose reductions after Dermulin™ administration of insulin vs. injection.

FIGS. 3A and B are graphical presentations of the % reduction in blood glucose limits effected by delivery of insulin by intestinal (Intesulin™-na) vs. subcutaneous injection vs. control using 2 different formulations.

FIG. 3A is a graphical representation of % reduction in blood glucose levels effected by insulin delivered transdermally (Dermulin™-C) vs. Subcutaneous injection vs. control; FIG. 3B is a graphical presentation of the % reduction in blood glucose limits effected by delivery of insulin by intestinal (Intesulin™-na) vs. subcutaneous injection vs. control using 2 different formulations (2 insulin doses=6.25 unites or 12.5 units). The controls—have no insulin, but have the same other ingredients. Injection—has the same insulin dose (Humalog) by subcutaneous injection.

FIG. 4A is a graphical representation of % reduction in blood glucose levels effected by insulin delivered transdermally with Dermulin™ vs. control, na; FIG. 4B shows (Dermulin™-WG) vs. Azone (Dermulin™-Azone) vs. control.

FIG. 4A and B are graphical representations of the % in blood glucose reduction effected by delivery of insulin via intestinal (Intesulin™-C) vs. subcutaneous injection vs. control.

FIG. 5 is a graphical representation of % blood glucose reduction effected by insulin delivery intragastrically (Gastrisulin™-A) vs. subcutaneous injection vs. control.

FIG. 6 is a graphical representation of % reduction of blood glucose levels effected by insulin delivered transdermally (Dermulin™C) vs. subcutaneously. “C” formulations were more potent for this type of delivery.

FIG. 6A is a graphical representation of insulin levels over time when insulin is in the form of controls vs. animals treated with Intesulin™-C and control vs. animals treated with Intesulin-No.

FIG. 6B shows insulin levels over time vs. subcutaneous injection vs. control levels.

FIG. 7 is Intesulin insulin bioavailability. Controls are treated the same but without insulin. FIG. 7 is a graphical representation of % reduction of blood glucose levels effected by insulin delivered transdermally (Dermulin™ Azone™) vs. Dermulin™-C vs. control.

FIG. 8 is a representation of insulin levels after transdermal delivery.

FIG. 9 is a representation of levels in Musulin™ vs. control.

FIG. 10 is a representation of insulin levels after Intesulin™.

FIG. 11 is a representation of insulin levels after Intesulin-B™ in the A. the jugular vein and B. the portal vs. the jugular vein.

FIG. 13 is a representation of insulin levels after Intesulin™-na via portal vs. jugular vein.

FIG. 14 is a graphical representation of blood insulin in intesulin-na via the portal vein.

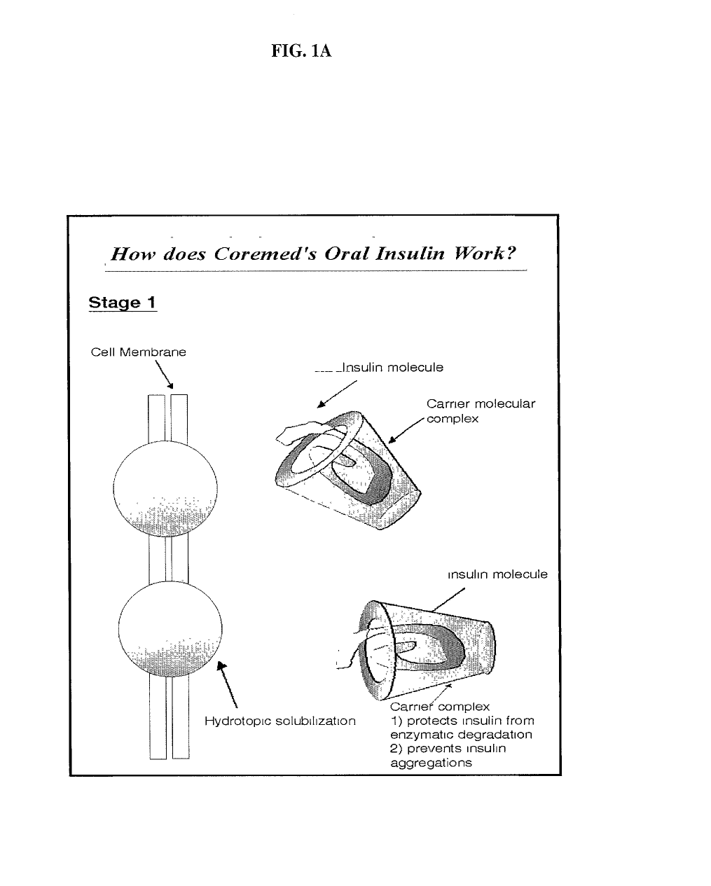

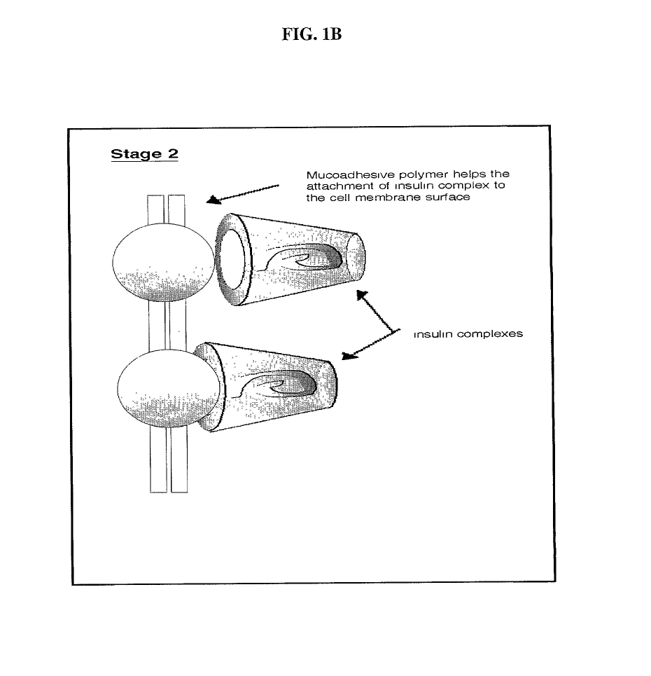

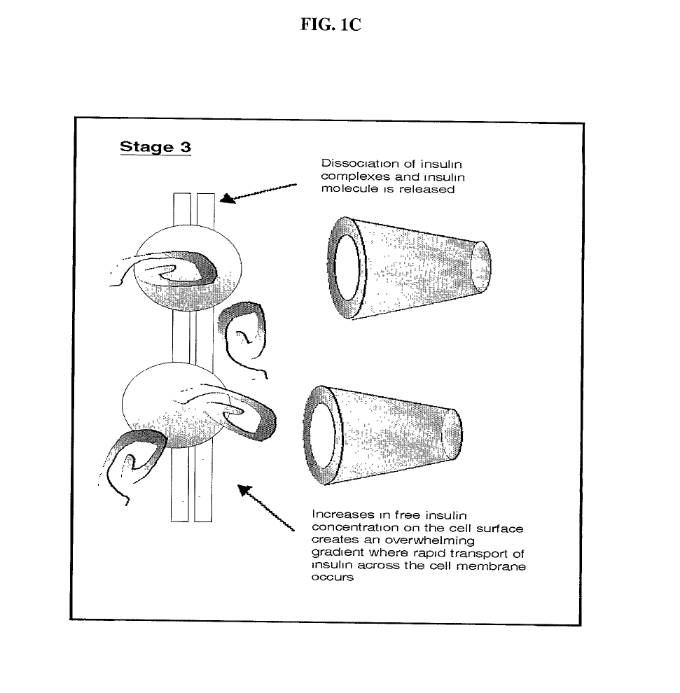

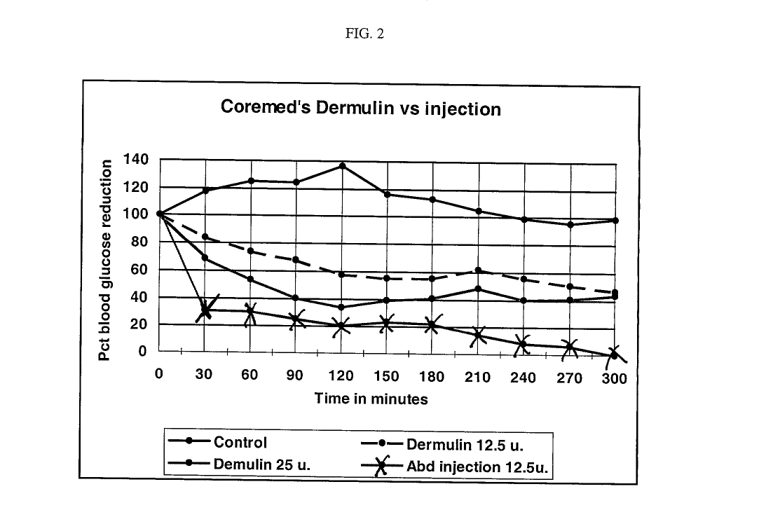

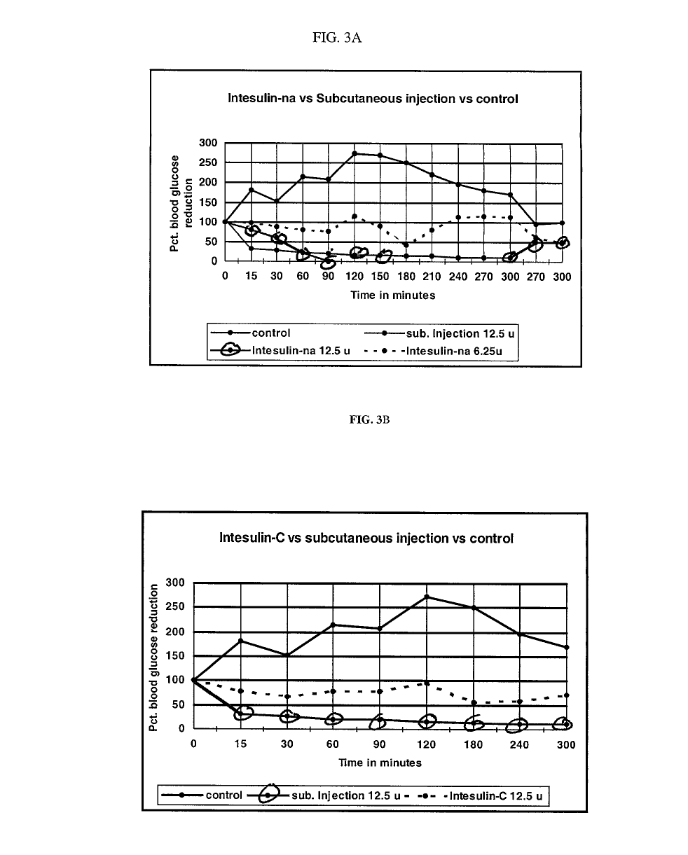

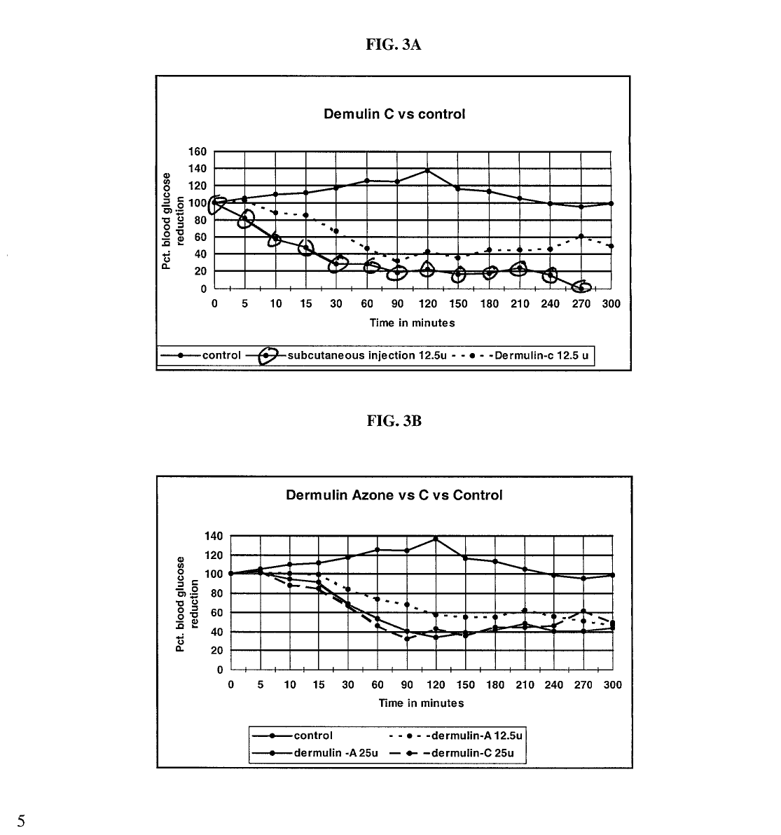

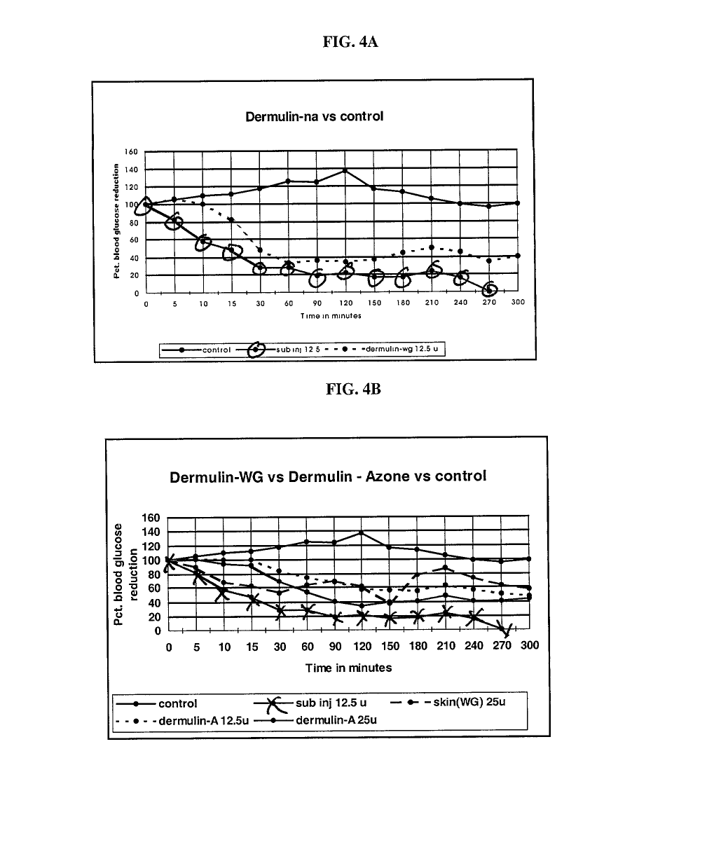

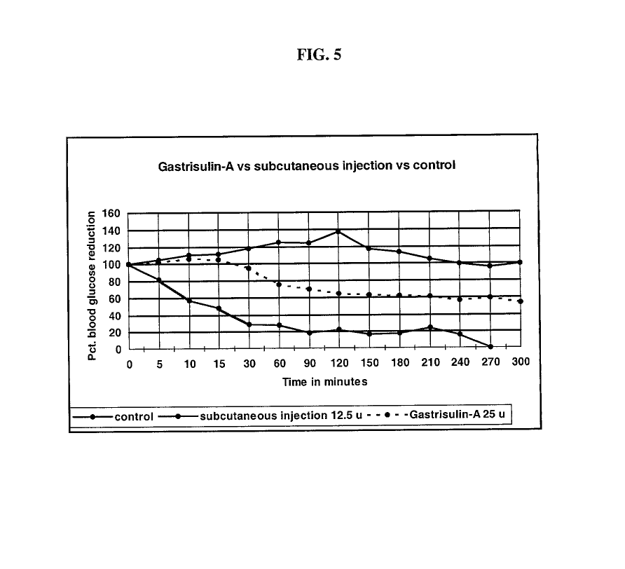

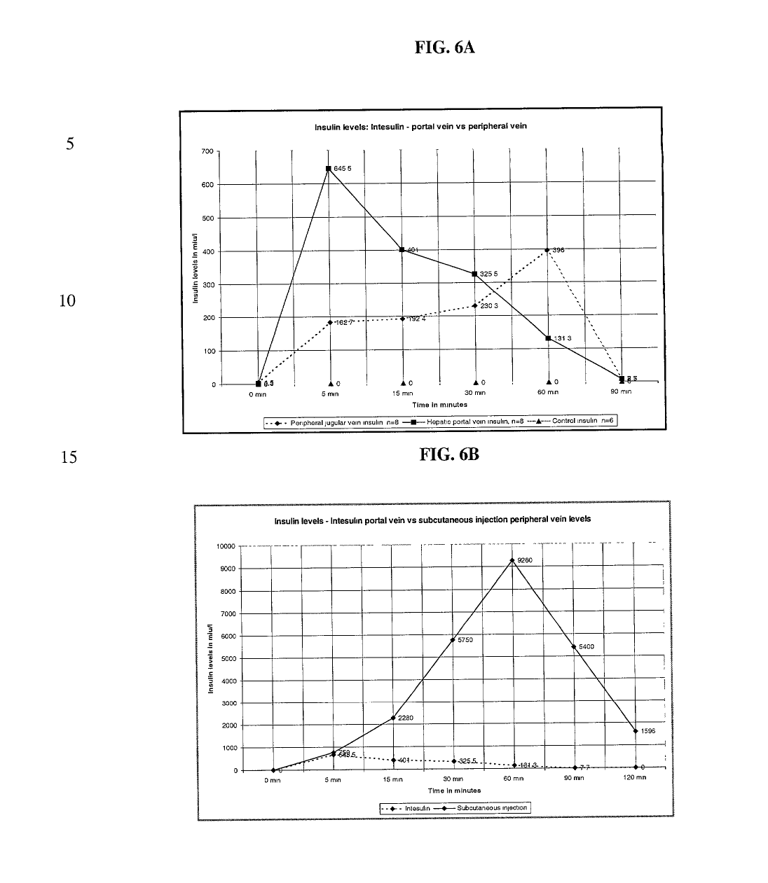

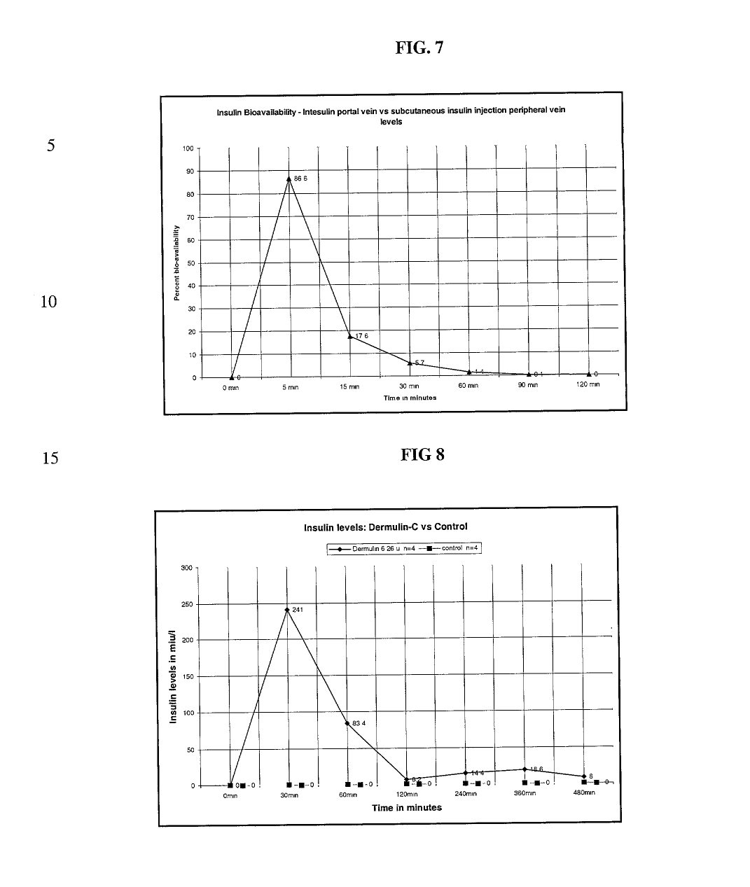

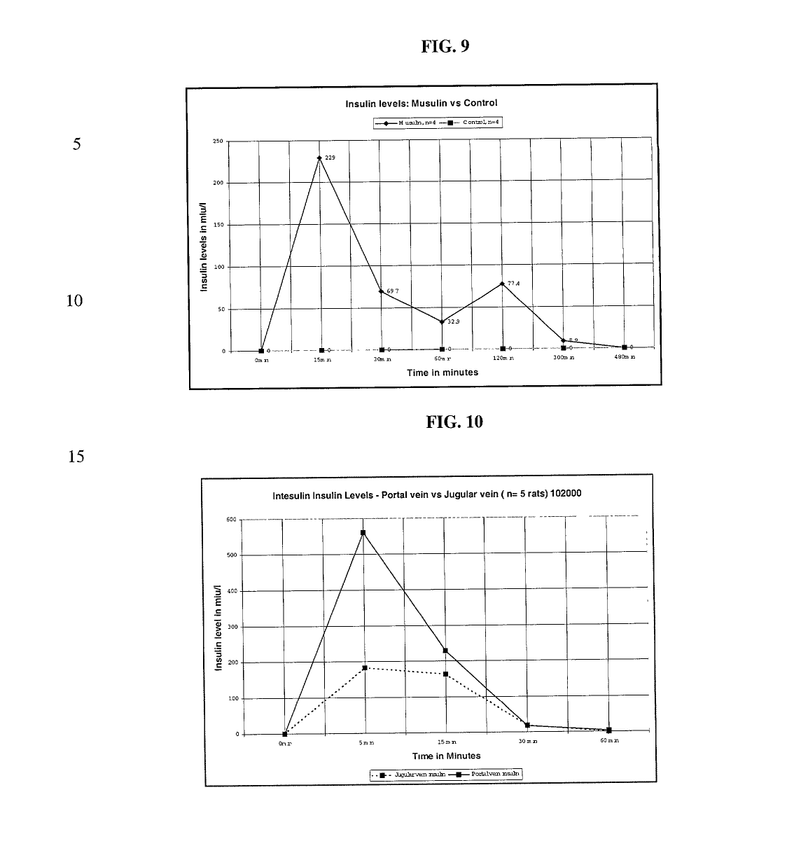

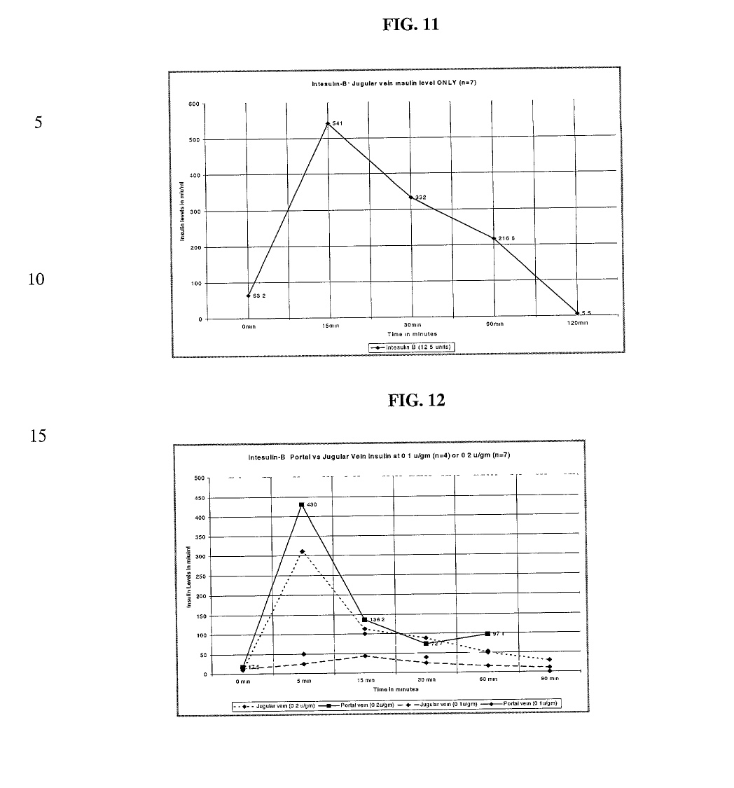

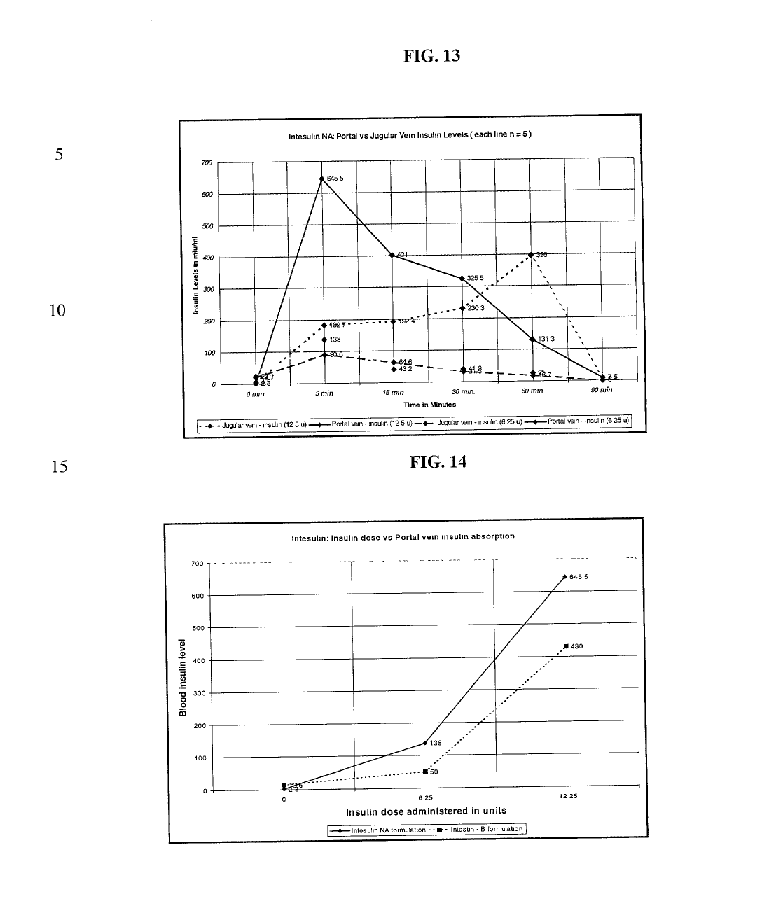

### DESCRIPTION OF THE INVENTION

Beneficial aspects of the methods and compositions of the present invention include delivery of significant amount of insulin transdermally without the use of enhancers.

1. Cost Analysis:

The cost of developing aerosol inhalers to deliver either pulmonary insulin or oral insulin is very expensive. Not only does the equipment have to be very reliable and precise, but in addition, the medication dosing delivered has to be exact. Oral or transdermal formulations of the present invention are estimated to cost 0.04 cents or less per dose in materials. Assuming the inhaled or oral insulin cost per month to the patient is about $100 (Both Advandia or Actos oral hypoglycemic agents cost $80-100 per month), the cost of the present formulation is about $0.25 per month (the patient takes medication twice a day). In volume production, the cost should go down by 50%. There will be a very significant cost advantage for the present formulations compared to all other current insulin prototypes under development.

2. Stability Analysis:

Both inhaled insulin or oral insulin spray requires insulin to be in dry powder form. In this form it is sensitive to temperature, moisture and humidity, because the drug may aggregate, precipitate or migrate over time. In comparison, the formulation of the present invention is in stable liquid form.

3. Irritability Analysis:

Medication powder directly sprayed into the airway or oral cavity can potentially irritate the mucosa membrane, cause coughing, and irritability. In disease state such as allergic rhinitis, common cold, asthma, tonsillities or nausea, the delivery of aerosol could be even more difficult. Many oral insulin formulations contain bile-salts or bile derivatives which are potentially very irritating to the mucous membranes. Although spraying small amounts can minimize the irritation, the side effects over long time of this form of administration are still unknown. Formulations of the present invention can be delivered transdermally, swallowed or transmucosally.

4. Convenience Analysis:

The device for inhaled insulin is several inches in length making it inconvenient to carry and use. Size can decrease compliance. Another problem is that oral insulin spray can occasalonally misfire. In contrast, the formulation of the present invention is a liquid or gel capsule which can be carried in small bottles and be delivered with precision.

5. Potency Analysis:

The reported poetency of inhaled insulin of oral insulin in literature is between 15%-50% (Dyschendra, 1998). The most up-to-date oral insulin in clinical trial is reported to lower blood glucose by about 30% and requires some 70 units. Transdermal insulin of the present invention lowers blood glucose by 60-80% and the oral insulin by 60-96%. Transdermal insulin has 60-70% efficacy compared to identical doses of insulin injection.

6. Pharamcologic Profile Analysis:

Inhaled insulin has quick onset of action and relatively short duration. Oral insulin has a lag time of 60 minutes before onset. Trasndermal insulin of the present invention has a quick onset of 15 minutes comparable to inhaled insulin. Its action lasts more than 8 hours and much longer than inhaled or the other oral insulin reported. Oral insulin can last at least 3-5 hours.

7. Ingredients:

Inhaled insulin contains dry powdered insulin and safe recipients such as albumin. Oral insulins may contain bile-salt or derivatives or mid-chain triglycerides, that can be irritating to the mucous membrane if sprayed in large amounts.

For compositions of the present invention, four preferred formulations involve each route of delivery—transdermal, oral-mucosa, intragastric and intestine. One fomulation contains an enhancer (Azone) which can be irritating to skin if in large amounts, the enhancer has been used in two of the FDA approved Orphan Drug products (1. methodrexate; 2. estrogen). Three other formulations contain no Azone. All other ingredients are FDA approved either GRAS and are being used in some of the current formulations on the market.

Formulations of the present invention (see Materials and Methods) are by far the lowest in cost of materials and manufacturing. They are more convenient to carry, easier to administer and well tolerated. More importantly, they are more potent and have an excellent pharmacologic profile when compared to any of the known formulations on the market. There is a choice of 4 different formulations: transdermal, oral-transmucosa, intragastric and intestinal.

### EXAMPLES

The following examples exemplify the present invention.

### Example

### The Hypoglycemic Efficacy of Insulin Oral Delivery Compared to Subcutaneous and Intraperitoneal Injections in Rats

Purpose:

To evaluate and compare the relative hypoglycemic efficacy between an oral insulin (Musulin™ 8100, 40 i.u./ml), intraperitoneal and subcutaneous insulin injections in nondiabetic rats.

Methods:

All rats weighed 50-80 gms, age of the rats was 1 month. Group 1 (control—placebo): 6 rats; Group 2 (Musulin™): 6 rats; Group 3 (Musulin™): 5 rats; Group 4 (Musulin™): 5 rats; Group 5 (intraperitoneal injection): 5 rats; and Group 6 (subcutaneous injection): 5 rats. All rats were fasted 2 hrs before experiments. They were lightly anesthesized by ether. Neck skin was dissected and the jugular vein was exposed. 0.1 ml of blood was drawn at times 0, 5, 10,15, 30, 60, 90, 120, 150, 180, 210, 240, 270, 300 minutes for glucose measurement by Abbott's Precision glucose monitor. In order to ensure the maximal exposure and absorption of Musulin™ in the rats' tiny oral cavities, the rats were fed constant amounts at preset time periods. Group 2, 5 and 6 each received insulin administration at 1.67 unit every 5 minutes. Group 3 received 3.33 units and Group 4 received 6.67 units every 5 minutes. The end-point of administering insulin is when the blood glucose dropped to 50% of the time-0 measurements.

Statistical Analysis:

All data were analysed using SAS system, Version 6.12. in 2 ways: 1) Simultaneous comparison of multiple groups by Dunnett's t test at 5% significance level. 2) Comparison of slopes of the glucose reduction rate by repeated measures analysis of variance method by the “Mixed Procedure” of the SAS system software.

Data:

1) Statistically significant onset of action: Group 5,6 at 10 minutes; Group 3,4 at 60 minutes and Group 2 at 90 minutes.

2) Rate of glucose reduction at the maximal slope: Group 1: +0.14 mg/min.; Group 2: −0.48 mg/min; Group3: −0.66 mg/min.; Group 4: −0.72 mg/min.; Group 5: −0.54 mg/min. and Group 6: −0.51 mg/min. Results of Groups 2, 3, 4, 5, 6 were not statistically significantly different from each other.

3) Percent mean reduction of glucose compared to initial levels at 60, 120 and 300 minutes were: Group 1: 124.3 (s.d. 20.8), 116.5 (s.d. 29.6), 98.2 (s.d. 28); Group 2: 98.5 (s.d. 45.8), 55.5 (s.d. 37.3), 40.7 (s.d. 25.4); Group 3: 54.6 (s.d. 29.6), 20.4 (s.d. 15.3), 40.5 (s.d. 15); Group 4: 54 (s.d. 20.9), 22 (s.d. 16), 36 (s.d. 48.4); Group 5: 30.4 (s.d. 3.8), 18.7 (s.d. 15.2), 0 (s.d. 0) and Group 6:39.8 (25.5), 25.5 (s.d. 31.7), 41.1 (s.d.-). The time in minutes for 50% rats to achieve 50% glucose reductions were: Group 2: 120, Group 3: 45, Group 4: 60, Group 5: 13 and Group 6: 15. Death from hypoglycemia within 300 minutes: Group 1: 0%, Group 2: 33%, Group 3: 40%, Group 4: 80%, Group 5: 80% and Group 6: 100%.

Summary:

The significant onset of action for the groups given higher oral insulin doses is at 60 min. and the lower dose group is at 90 min., suggesting a dose dependent effect. Musulin™ exerts a similar rate of lowering glucose as the intraperitoneal and subcutaneous injections at the maximal slope from 0-180 min. Statisically significant hypoglycemia lasted at least 4.5 hours. Although mean blood glucose levels remained low beyond 5 hours, statistical analysis cannot be validated beyond 4.5 hours due to the data bias from animal death from hypoglycemia and the rebounding blood glucose in the surviving animals.

Conclusion:

After a delayed onset of action between 60-90 minutes, Musulin™ exerts a hypoglycemic action identical to both the injection groups. A significant number of rats died from hypoglycemia due to Musulin™. Hypoglycemia lasts at least 4.5 hours. The mean blood glucose levels at 120 minutes in the Musulin™ groups were between 61% to 96.1% lower than the placebo group. Musulin™ is a potent oral insulin.

### Example 2

A Rapid on-set Transdermal Insulin with 60-70% Efficacy as the Subcutaneous Insulin Injection in Rats

Purpose:

To evaluate and compare the relative hypoglycemic efficacy and glucose response between a transdermal insulin (Dermulin™ 8200) and subcutaneous insulin injection in nondiabetic rats.

Methods:

All rats weighed 50-80 gms, aged 1 month. Group 1 (control—placebo): 6 rats, Group 2 (Dermulin™, 12.5 u): 5 rats, Group 3 (Dermulin™, 25 u): 5 rats, Group 4 (subcutaneous injection): 5 rats.

Protocol:

All rats were fasted 2 hrs before experiments. They were lightly anesthesized by ether. Neck skin was dissected and jugular vein was exposed. 0.1 ml of blood was drawn at time 0, 5, 10, 15, 30, 60, 90, 120, 150, 180, 210, 240, 270, 300 minutes for glucose measurement by Abbott's Precision glucose monitor. Hair was shaved from the abdomen. Group 2 and Group 3 had 12.5 u and 25 u of Dermulin™ applied to the abdominal skin respectively. Group 4 received one single injection of 12.5 u of insulin subcutaneously.

Statistical Analysis:

All data were analyzed using SAS system, Version 6.12. in 2 ways: 1) Simultaneous comparison of multiple groups by Dunnett's t test at 5% significance level. 2) Comparison of slopes of the glucose reduction rate by repeated measures analysis of variance method by the” Mixed Procedure” of the SAS system software.

Data:

1) Statistically significant onset of action: Group 4 at 5 minutes; Group 3 at 15 minutes and Group 2 at 30 minutes.

2) Significant hypoglycemia lasted up to 180 minutes. Mean blood glucose levels remained low beyond 5 hours, statistical analysis cannot be validated beyond 3 hours due to the data bias from animal death from hypoglycemia and the rebounding blood glucose in the surviving animals.

3) Rate of glucose reduction at the slope between 30-120 min.: Group 1: +0.01 mg/min.; Group 2: −0.41 mg/min; Group 3: −0.58 mg/min.; Group 4: −0.5 mg/min. The rate is statistically different between Group 2 and Group 4, but insignificant between Group 3 and Group 4. 4) Percent of glucose reduction compared to initial levels at 60, 120, 180 and 300 minutes were: Group 1: 124.3 (s.d., 20.8), 116.5 (s.d. 29.6), 98.2 (s.d. 28), 98.2 (s.d. 28); Group 2: 74 (s.d. 24.8), 57.5 (s.d. 21.8), 55.3 (s.d. 15.5), 58.5 (s.d. 23.5); Group 3: 53.4 (s.d. 16.7), 34.2 (s.d. 17.4), 51.1 (s.d. 22.7), 54.5 (s.d. 10.3); Group 4: 28 (s.d. 13.4), 21.9 (s.d. 13), 17.2 (s.d. 14.1), 0.

Summary:

Statistically significant onset of action for the injection group occurred at 5 minutes., 25 u of Dermulin™ at 15 minutes and 12.5 u of Dermulin™ at 30 minutes. Efficacy (E) is defined as the ratio of percent glucose reduction compared to placebo by Dermulin™ divided by the percentage by subsutaneous injection. E of 12.5 U of Dermulin at 5, 10, 15, 30 min. were 19.8,42.7,52.4,66.4. After 30 min., the E plateaued at 60-70% until 240 min. 25 u of Dermulin has similar rate in lowering glucose as the subcutaneous injection between 15-120 min. Statistically significant hypoglycemia lasted at least 3 hours.

Conclusion:

Dermulin™ is an potent transdermal insulin formulation. The mean blood glucose levels at 120 minutes in the Dermulin™ groups were between 59.3% to 82.3%, significantly lower than the placebo group. Dermulin™ has rapid onset of action at 15-30 min. which is dose dependent. The calculated efficacy of Dermulin™ compared to the identical dose by subcutaneous injection is 60-70%.

### Example 3

### Blood Glucose Response to Transdermal Insulin Administration and its Hypoglycemic Potency in Comparison to Intraperitoneal Insulin Injection in Rats

Purpose:

To evaluate and compare the relative hypoglycemic potency and glucose response between a transdermal insulin (designated by the inventors Dermulin™ 8200) and intraperitoneal insulin injection in nondiabetic rats.

Methods:

All rats weighed 50-80 gms, aged 1 month. Group 1 (control—placebo): 6 rats, Group 2 (Dermulin™, 12.5 u): 5 rats, Group 3 (Dermulin™, 25 u): 5 rats, Group 4 (intraperitoneal injection): 5 rats.

Protocol:

All rats were fasted 2 hrs before experiments. They were lightly anesthesized by ether. Neck skin was dissected and jugular vein was exposed. 0.1 ml of blood was drawn at time 0, 5, 10, 15, 30, 60, 90, 120, 150, 180, 210, 240, 270, 300 minutes for glucose measurement by Abbott's Precision glucose monitor. Hair was shaved from the abdomen. Group 2 and Group3 had 12.5 u and 25 u of Dermulin™ applied to the abdominal skin respectively. Group 4 received one single injection of 12.5 u of insulin intraperitoneally.

Statistical Analysis:

All data were analyzed using SAS system, Version 6.12. in 2 ways: 1) Simultaneous comparison of multiple groups by Dunnett's t test at 5% significance level. 2) Comparison of slopes of the glucose reduction rate by repeated measures analysis of variance method by the “Mixed Procedure” of the SAS system software.

Data:

1) Statistically significant onset of action: Group 4 at 5 minutes; Group 3 at 15 minutes and Group 2 at 30 minutes.

2) Significant hypoglycemia lasted up to 180 minutes. Mean blood glucose levels remained low beyond 5 hours, statistical analysis cannot be validated beyond 3 hours due to the data bias from animal death from hypoglycemia and the rebounding blood glucose in the surviving animals.

3) Rate of glucose reduction at the slope: Group 1: +0.01 mg/min.; Group 2: −0.41 mg/min; Group3: −0.58 mg/min.; Group 4: −0.5 mg/min. Statistically, the rate is different between Group 2 and Group 4, but insignificant between Group 3 and Group 4. 4) Percent of glucose reduction at 60, 120, 180 and 300 minutes were: Group 1: 124.3 (s.d., 20.8), 116.5 (s.d. 29.6), 98.2 (s.d. 28), 98.2 (s.d. 28); Group 2: 74 (s.d. 24.8), 57.5 (s.d. 21.8), 55.3 (s.d. 15.5), 58.5 (s.d. 23.5); Group 3: 53.4 (s.d. 16.7), 34.2 (s.d. 17.4), 51.1 (s.d. 22.7), 54.5 (s.d. 10.3); Group 4: 26.1 (s.d. 13.4), 16.5 (s.d. 13), 25.8 (s.d. 14.1), 27.8 (s.d. 33.6).

Summary:

The significant onset of action for the injection group occurred at 5 minutes., 25 u of Dermulin™ at 15 minutes and 12.5 u of Dermulin™ at 30 minutes, suggesting a dose dependent effect. 25 u of Dermulin™ has comparable strength in lowering glucose at similar rate as the intraperitoneal injection of insulin at 12.5 u. The rate of glucose reduction in the 12.5 u Dermulin™ group is 82% (−0.41/−0.5=0.82) of the rate occurred in the 12.5 u intraperitoneal insulin injection group. Statisically significant hypoglycemia lasted at least 3 hours.

Conclusion:

Dermulin™, a transdermal insulin has 82% of comparable hypoglycemic strength in the rate of glucose reduction as the intraperitoneal insulin injections. It has a quick onset of action at 15-30 minutes. Its hypoglycemic action sustains at least 3 hours or more. The mean blood glucose levels at 120 minutes in the Dermulin™ groups were between 59.3% to 82.3% lower than the placebo group.

### Example 4

Oral insulin (Intesulin-B) showed marked effects by lowering triglyceride levels in non-diabetic rats. Triglyceride was lowered by as much as 60% in 24 hours after treatment. Animals were allowed to feed after 8 hours. The study was continued up to 48 hours.

Glucose levels in the same experiment:

| | | |
|---|---|---|
| At time | 0 min: | 100% (no change). |
| | 15 min: | 79.9% (decrease 79.9% compared to control). |
| | 60 min: | 139.9% (decrease by 139.9% comparted to |
| | | control). |
| | 120 min: | 216.9% (decrease by 216.9% because control's |
| | | glucose went up). |

Materials and Methods

Summary of Intesulin-B Formulation

1) All ingredients are US FDA approved;

2) It is extremely rapidly absorbed;

3) The peak Insulin absorption occurs at 5 minutes after administration. Experiments were repeated and confirmed in no less than 7 minutes.

4) Intesulin-B achieved similar therapeutic potency as subcutaneous injection.

5) It is slower onset in action than subcutaneous injection. The onset of hypoglycemic action is 15-60 minutes.

6) Its insulin pharmacokinetic match the finding in normal human physiology.

7) It has an effective and sustained hypoglycemic action without peripheral hyper insulinemic.

8) It demonstrated its superiority in overcoming enzymatic degradation over many hours of glucose the linear slope of glucose reduction in diabetic animals reduction in diabetic animals.

9) It has an excellent bioavailability profile.

10) It is many times more potent in it hypoglycemic action than subcutaneous injection (AUC glucose/AUC insulin).

11) It lowers triglyceride level by 60% in 24 hours.

12) Insulin peak absorption and total insulin bioavailability (AUC) in severe diabetic animals are 3-4 times higher than in non-diabetic control.

13) Severe diabetic animals absorbs Cormed's oral transmucosal insulin much better than in non-diabetic control.

14) Intesulin-B contains no irritable ingredients. Most of them are GRAS materials.

15) Number of animal studied as of November, 2000:258

Insulin for All Formulations

1. Alpha Cyclodextrin 0.2% (0.1-2%)—BCD—Hydroxypropyl BCD 1%

2. Sodium Lauryl Sulfate 0.5% (0.2-1%)

3. Urea 1.5% (0.5-2%)

4. Lispro insulin

5. Carboxymethycellulose 0.1% (0.05-0.5%)

6. Hydroxypropyl Methycellulose 0.2% (0.05-1%)—the final solution is about 5 times or more normal viscosity in humans

7. Prolylene Glycol 1-5% (0.5-10%)

8. Modifiers (boosters) of the formulations:

a. Capsaicin 0.05%: In the transdermal and intragastric delivery. Capsaicin has a similar chemical structure to Azone (Laurocapram™) which is reported to be a potent transdermal enhancers. The formulation including Capsaicin gives better insulin absorption in transdermal and intragastric delivery than other formulations.

b. Urea Peroxide 0.2%: This is a booster for additional insulin absorption. Urea peroxide will break down into urea, water molecules and oxygen. Additional water molecules help to free insulin molecules from the insulin-CD complexes. Additional oxygen molecules may help active transport of insulin in the ATP membrane active transport.

c. Winter-green oil is both a hydrophilic and lipophilic agent. It also prevents rapid drying effect on the transderaml formulation and preserves the layer of moisture.

d. Carbomer −970 at 0.1% is known to deactivate intestinal exzymes to prevent excess insulin degradation. It can also be used as viscosity control agent.

e. Lispro insulin solution: Insulin Lispro, rDNA origin is made by Eli Lily. The insulin solution is prepared according to Eli Lily's formulation. Zinc-insulin lispro crystals are dissolved in a clear aqueous fluid. Each milliliter of lispro insulin contains 100 units, 16 mg glycerin, 1.88 mg dibasic sodium phosphate, 3.15 mg m-cresol, zinc oxide content is adjusted to provide 0.0197 mg zinc ion, trace amounts of phenol and water.

f. Urea, CH4N20, molecular weight 60. Urea causes dry skin cells to “unpack” and expose their water binding sites, thus enabling the cells to absorb and retain additional moisture, which in the present invention will include insulin. This action is known hydrotopic solubilization. It is like taking the plastic wrapper off a sponge so it can absorb better. It is a “humectant” with moisturizing effect. It actively replenishes moisture to the surface of the absorptive membrane.

g. Alpha-Cyclodextrin (CD)—C36H60O30, molecular weight 972.852 daltons. Cyclodextrin was known and described by Villiers in 1891. Structurally, CD consists of 6, 7, or 8 (designated alpha, beta, gamma) D-glycosidic linkages. The more stable three dimensional molecular configuration for these non-reducing cyclic oligosaccharides takes the form of a toroid with the upper (large) and low (smaller) opening of the toroid presenting secondary and primary hydroxyl groups. The interior of the toroid is hydrophobic. It is the interplay of atomic (Van der Waals), thermodynamic (hydrogen bonding), and solvent (hydrophobic) forces that accounts for the stable complexes that may be formed with insulin while in the apolar environment of the CD cavity.

Dissociation is usually a rapid process, most often caused by the sudden rapid increase in the number of water molecules outside the cavity. The concentration gradient created becomes overwhelming and the insulin molecue is displaced. Unable to find relatively scarce CD molecules to reform the complex, insulin exists free in solution, depending on the amount of dilution and concentration of CD.

Therefore, CD can enhance the insulin aqueous solubility and protect insulin in their micro-environment from enzymatic attack and degradation, create and maintain stable homogeneous distribution in a harsh environment, resulting in greatly improving the absorption and bioavailability of insulin, preventing aggregation (Shao, 1992), prevent insulin degradation by proteolytic enzymes (Irei, 1992), and remove calcium, widening the tight junctions and increase paracellular transport.

CD consists of natural or chemically modified CD. Natural CD are alpha, beta and gamma. Chemical modified CD includes hydroxylpropyl beta CD, sulfated CD, acetylated beta CD, cationic beta CS and cationic gamma CF. Hydroxylpropyl beta CD has been approved for specific formulation by the US FDA. Alpha and beta CDS are approved by the regulatory agences in Europe and Japan. Some beta CDS are included in certain food products. The enhancing effect is: dimethyl beta CD>alpha CD>dimethyl gamma CD>hydroxypropyl beta CD>hydroxypropyl alpha CD>beta CD. But dimethyl beta CD can substitute for alpha CD, although the former is weaker. Concentration of the former can be increased.

Hydroxypropyl beta CD and beta CD can substitute for alpha CD, although the former is weaker. Concentration of the former can be increased.

h. Sodium Lauryl sulfate (151-21-3) (SLS): C12H25NaO4S, molecular weight. 288.37687. SLS acts as surfactants. It increases the solubilization of insulin perturbation of membrane layers, with increased transport and extraction of membrane proteins and lipids.

i. Hydroxypropyl methycellulose (9004-65-3) is used as a viscosity control agent in the formulation.

j. Carboxymethylcellulose (9000-11-7) (CM). CM is used in many consumer and pharmaceutical products. It is one of the most mucous bioadhesive agent. For example, a bioresorbable membrane made of sodium hyaluronate and carboxymethycellulose, has been reported to prevent peritoneal adhesion (Swiss Surgery, 1999). CM has been reported to widen the cellular junctions and improve absorption of certain compounds. It is also used as a lubricant and cushing agent in Celluvisc t.m. for the eyes. It is a common agent used in detergent and soaps.

k. Propylene glycol improves solubility and stabilization of the solution.

The composition of the ingridients in this invention will produce an insulin solution with pH 7.4. This is a very important parameter achieved without the use of buffering. Ritschel (1989) demonstrated buccal insulin absorption to be greatest at pH 7.5 when absorption was measured at different pH levels.

Results:

The results in animal experiments using the formulation of the present invention are dramatic.

The formulation allows the fastest insulin peak absorption ever reported in literature. Its peak insulin absorption in the intestine is 5 minutes, oral-transmucosal is 15 minutes and transdermal is 30 minutes. It is faster than subcutaneous insulin injection in control experiments which is 60 minutes.

The formulation is extremely potent, capable of lowering the blood glucose from 60 to 100% depending on the dose and site of application. The most potent is intestinal delivery, followed by oral-transmucosal, intragastric and transdermal.

The onset of hypoglycemic action is very rapid, that is, within 15 to 60 minutes depending on the site of delivery.

The amount of insulin absorbed into the blood stream is one of the highest reported in the literature.

The insulin profile of intestinal insulin delivery matches that in normal human physiology. That is, the peak insulin absorption occurs in 5-15 minutes and the insulin level returns to normal control level within 2 hours.

Method of Preparing a Formulation for the Present Invention [100 ml (25 units of Lispro™ Insulin per ml)]:

1. Dissolve 0.1 gm of carboxymethylcellulose at 60 C with 5 ml water. Cool solution to room temperature.

2. Dissolve 0.2 gm of hydroxymethycellulose in 5 ml of water at 20 degree temperature.

3. Dissolve 0.2 gm of alpha cyclodextrin in 3 ml of water at 20 degree temperature.

4. Dissolve 0.2 gm of sodium lauryl sulfate in 10 ml water at 20 degree temperature.

5. Dissolve 1 gm of urea in 3 ml water at 30 degree temperature.

6. Mix 25 ml (2500 units) of Lispro™ insulin solution with the alpha cyclodextrin solution at 20 degree temperature.

7. Mix the carboxymethylcellulose solution, the urea solution and the sodium lauryl sulfate solution together at room temperature.

8. Mix the Lispro-alpha cyclodextrin solution to the carboxymethylcellulose-urea-sodium sulfate solution together at room temperature.

9. Stir well.

10. Add the hydroxymethlcellulose solution, ad 2 ml propylene glycol solution to the solution containing insulin.

11. Add water to the final solution to the desirable concentration.

12. Stir well.

13. Add desirable amount of modifier such as Capsaicin to the final solution for transdermal formulation.

14. Add 0.2 gm of urea peroxide in 3 ml of water at 20 degree. Mix it with the final solution.

### DOCUMENTS CITED

A M A, Diabetes Care. (1998) 21: 296-309.

Canovan et al. (1997) Diabetologia 40(10): 1125-1134.

Damge, C. (1997) J. Pharm Sci. 86: 1403.

Dyschendra (1998) Critical Reviews in Therapeutic Drug Carrier Systems 15(6):629-670.

Irei, T. (1992) Int. J. Pharm. 84:129.

Ritschel, W A (1989) Res. Comm. Chem. Pathol. Pharmocol. 63:53-67.

Shao, A. (1992) Pharm. Res. 9:1157-1163.

Swiss Surgery (1999) 1:23-26.

Yamasaki, Y (1981) Can. J. Physiol.

Yamasaki, Y (1980) Diabetic Care.

I claim:

1. A composition comprising
(a) insulin;
(b) a carrier molecule; and
(c) a binding agent to facilitate attachment of insulin to a cell membrane.

2. A method for delivering insulin to a person in need thereof, said method comprising:
(a) obtaining insulin in a formulation suitable for oral delivery; and
(b) delivering an effective amount of the formulation to the person in need thereof, wherein delivery does not require injection.

3. The method of claim 2, wherein the delivery is intragastric or intestinal.

4. The method of claim 2, wherein the delivery is transdermally.

5. A method for preparing a composition of insulin, for delivery without injection said method comprising:
a. dissolving 0.1 gm of Carboxymethylcellulose at 60° C. with 5 ml water;
b. cooling the solution to room temperature;
c. dissolving 0.2 gm of Hydroxymethycellulose in 5 ml of water at 20° C.;
d. dissolving 0.2 gm of Alpha Cyclodextrin (0.1%) or Hydroxy B-Cyclodextrin (0.5%) in 3 ml of water at 20° C.;
e. dissolving 0.2 gm of Sodium Lauryl Sulfate in 10 ml water at 20° C.;
f. dissolving 1 gm of urea in 3 ml water at 30° C.;
g. mixing 25 ml (2500 units) of Lispro insulin solution with the Alpha Cyclodextrin Hydroxy B-Cyclodextrin solution at 20° C.;
h. mixing the Carboxymethylcellulose solution, the urea solution and the Sodium Lauryl Sulfate solution together at room temperature;
i. mixing the Lispro-Alpyha Cyclodextrin (Hydroxy B-Cyclodextrin) solution with the Carboxymethylcellulose-Urea-Sodium Sulfate solution at room temperature.
j. adding the Hydroxymethlcellulose solution, and 2 ml of the Propylene Glycol solution to the solution containing insulin;
k. adding water to the final solution to a desired concentration;
l. adding the desired amount of modifier to the final solution for a transdermal formulation; and
m. adding 0.2 gm of urea peroxide in 3 ml of water at 20 degree and adding it to the formulation.
